(19) United States
(12) Patent Application Publication
(10) Pub. No.: US 2024/0366412 A1
(43) Pub. Date: Nov. 7, 2024

(54) APPARATUS FOR POSITIONING A LEG DURING SURGERY

(71) Applicant: Jesus Fajardo, Cucamonga, CA (US)

(72) Inventor: Jesus Fajardo, Cucamonga, CA (US)

(21) Appl. No.: 18/651,717

(22) Filed: May 1, 2024

Related U.S. Application Data
(60) Provisional application No. 63/463,079, filed on May 1, 2023.

(51) Int. Cl. A61F 5/37; A61G 13/00; A61G 13/12

(52) U.S. Cl. CPC A61F 5/3761; A61G 13/0036; A61G 13/1245

(57) ABSTRACT

A surgical positioning device may include a boot having a pin connected thereto and an adapter, the adapter comprising: a t-track, the t-track is formed from a first protrusion having a first flange located opposite a second protrusion having second flange, a housing, the housing is hingedly pivotable at a fulcrum, the housing having a chamber, the chamber of the housing is configured to receive the pin of the protrusion structure of the boot, wherein the housing is in communication with an outer wall surface of the t-track; and a guide, the guide having a first side located opposite a second side, the first side of the guide having a t-bolt, the t-bolt is configured to slidably traverse the t-track, the second side of the guide having a peg, the peg is configured to be received by an opening of a peg board.

### CROSS-REFERENCE TO RELATED APPLICATIONS

The present application claims priority under 35 USC 119(e) to U.S. Provisional Application No. 63/463,079 filed May 1, 2023, the entire contents of which is incorporated by reference.

### FIELD OF THE EMBODIMENTS

The field of the invention and its embodiments relate to a limb holder, such as a surgical leg positioner for supporting a limb of a patient, and related methods for attachment to a surgical table.

### BACKGROUND OF THE EMBODIMENTS

A total hip replacement, also referred to as a total hip arthroplasty (THA), is a commonly performed procedure indicated for the treatment of damage to the hip by arthritis, a fracture, and/or other conditions. A THA procedure is commonly characterized by damaged bone and/or cartilage of the hip being removed and replaced with prosthetic components. There are several approaches for hip surgery including anterior, anterolateral, lateral, and posterior approaches. The patient is usually placed in the supine or lateral position during surgery, depending on the surgical approach and surgeon preference. During anterior-based total hip arthroplasty, the patient is usually in the supine position. One of the difficulties of anterior-based total hip arthroplasty is obtaining adequate, stable, reproducible exposure of the hip for preparation of the bone and placement of the prosthetic implants.

One of the ways exposure to the hip is obtained and optimized is through manipulation of the lower extremity. Manipulation of the extremity can be accomplished by two methods: (1) an additional trained surgical assistant or (2) a specialized table or table attachment. Using an additional trained surgical assistant for the purpose of manipulating the extremity during surgery can be costly, and may further deplete limited human resources. Furthermore, it is difficult for an assistant to maintain the extremity perfectly stable for periods of time, especially since they often need to be in non-ergonomic positions in order to stay out of the way of the surgeon performing the procedure. To avoid these issues, special tables as well as special table attachments have been developed to maneuver and stabilize the lower extremity during surgery. However, many of these table attachments have inherent problems and limitations.

First, such table attachments or specialized tables can make it difficult for a surgeon to execute a manual prosthesis check to assess stability and range of motion, due to the way the lower extremity is secured and because the distal lower extremity is usually not sterile. Second, many table attachments and specialized tables necessitate that an additional assistant be utilized. This assistant must have significant training and experience operating the table attachment/specialized table so that the extremity does not experience excessive rotation or excessive force. Excessive rotation or excessive force can result in femoral fracture, which can be a catastrophic complication. Lastly, specialized tables and large table attachments can be large, costly, require additional storage space, and require maneuvering of equipment around the operating room in between surgeries. Small bed attachments, on the other hand, are limited in their ability to maneuver and stabilize the lower extremity.

To address the need for obtaining adequate, stable, and reproducible exposure of the hip during anterior total hip arthroplasty (and related hip procedures performed in the supine position) while also avoiding the limitations of currently available devices, a new and novel leg positioner is hereby proposed. This leg positioner is intended to be easily portable and compatible with most standard operating tables. It is intended to be sterilized and used by the surgeon during the operation to ensure appropriate position, appropriate rotation, and appropriate force is applied. This will help optimize exposure while minimizing the risk of iatrogenic injury such as fracture due to manipulation of the extremity by an unexperienced assistant. Furthermore, this leg positioner will eliminate the need for an extra trained surgical assistant to manipulate the lower extremity during surgery either directly or by operating a specialized table or table attachment.

Some leg positioning systems in today's market typically have a boot configured to receive a patient's foot. The boot is configured to slidably traverse a rod. The rod is connected to an axle slidably mounted on a curved track. The boot is configured to be rotated and/or positioned along the curved track. Although these free-standing leg positioning systems may be used with an existing operating table, they are large, heavy, and bulky. Further, these large leg positioning systems may obstruct direct access of a medical professional to a patient during use. As a result, necessitating these medical professionals with the burden of having to step over and/or navigate around the large and bulky leg positioning system during a procedure. It would be more desirable to have a leg positioning system that is smaller in size and less bulky as to not impede a medical professional from being able to perform a procedure.

### SUMMARY OF THE EMBODIMENTS

In accordance with the principles of the present invention, a surgical positioning device may include a boot having a pin connected thereto and an adapter, the adapter comprising: a t-track, the t-track is formed from a first protrusion having a first flange located opposite a second protrusion having second flange, a housing, the housing is hingedly pivotable at a fulcrum, the housing having a chamber, the chamber of the housing is configured to receive the pin of the protrusion structure of the boot, wherein the housing is in communication with an outer wall surface of the t-track; and a guide, the guide having a first side located opposite a second side, the first side of the guide having a t-bolt, the t-bolt is configured to slidably traverse the t-track, the second side of the guide having a peg, the peg is configured to be received by an opening of a peg board, and which also includes improvements that overcome the limitations of prior surgical leg positioners, is now met by a new, useful, and non-obvious invention.

In some aspects, the techniques described herein relate to a surgical positioning device, including: a boot, the boot including: a receiving structure, the receiving structure having a channel, the channel having a radiused surface configured to receive a distal portion of a limb of a user; a plate, the plate is connected to an end of the receiving structure, the plate having a first side located opposite a second side, the plate is oriented perpendicular to the channel of the receiving structure; and a protrusion structure, the protrusion structure connected to the second side of the plate, the protrusion structure having a pin; an adapter, the adapter including: a t-track, the t-track is formed from a first protrusion having a first flange located opposite a second protrusion having second flange; a housing, the housing is hingedly pivotable at a fulcrum, the housing having a chamber, the chamber of the housing is configured to receive the pin of the protrusion structure of the boot; wherein the housing is in communication with an outer wall surface of the t-track; and a guide, the guide having a first side located opposite a second side, the first side of the guide having a t-bolt, the t-bolt is configured to slidably traverse the t-track, the second side of the guide having a peg, the peg is configured to be received by an opening of a peg board.

In some aspects, the techniques described herein relate to a surgical positioning device, wherein the t-track having a locking mechanism.

In some aspects, the techniques described herein relate to a surgical positioning device, wherein the locking mechanism is a knob connected to a threaded rod.

In some aspects, the techniques described herein relate to a surgical positioning device, wherein the locking mechanism having a ratcheting element with a resilient member.

In some aspects, the techniques described herein relate to a surgical positioning device, further including a pegboard, the pegboard having at least one opening configured to receive the peg of the guide of the adapter.

In some aspects, the techniques described herein relate to a surgical positioning device, further including a clamp, the clamp is configured to connect the pegboard to at least a portion of a table.

In some aspects, the techniques described herein relate to a surgical positioning device, further including a first L-bracket having a first end and a second end, the first end of the first L-bracket is removably connected to the clamp, the second end of the first L-bracket having an opening, the opening of the second end of the first L-bracket is configured to receive a first end of a second L-bracket, a second end of the second L-bracket is configured to be received by the at least one opening of the pegboard.

In some aspects, the techniques described herein relate to a method of using a surgical positioning device, including: providing a surgical positioning system; providing a table; providing a pegboard, the pegboard overlaying the table; placing a distal end of a user's limb within a boot; and connecting the boot to the pegboard.

Clause 1. A surgical positioning device, comprising: a boot, the boot comprising: a receiving structure, the receiving structure having a channel, the channel having a radiused surface configured to receive a distal portion of a limb of a user; a plate, the plate is connected to an end of the receiving structure, the plate having a first side located opposite a second side, the plate is oriented perpendicular to the channel of the receiving structure; and a protrusion structure, the protrusion structure connected to the second side of the plate, the protrusion structure having a pin; an adapter, the adapter comprising: a t-track, the t-track is formed from a first protrusion having a first flange located opposite a second protrusion having second flange; a housing, the housing is hingedly pivotable at a fulcrum, the housing having a chamber, the chamber of the housing is configured to receive the pin of the protrusion structure of the boot; wherein the housing is in communication with an outer wall surface of the t-track; and a guide, the guide having a first side located opposite a second side, the first side of the guide having a t-bolt, the t-bolt is configured to slidably traverse the t-track, the second side of the guide having a peg, the peg is configured to be received by an opening of a peg board.

Clause 2. The surgical positioning device of clause 1, wherein the t-track having a locking mechanism.

Clause 3. The surgical positioning device of clause 2, wherein the locking mechanism is a knob connected to a threaded rod.

Clause 4. The surgical positioning device of clause 3, wherein the threaded rod having a plate connected to an end portion.

Clause 5. The surgical positioning device of clause 1, further comprising a pegboard, the pegboard having at least one opening configured to receive the peg of the guide of the adapter.

Clause 6. The surgical positioning device of clause 5, further comprising a clamp, the clamp is configured to connect the pegboard to at least a portion of a table.

Clause 7. The surgical positioning device of clause 6, further comprising a first L-bracket having a first end and a second end, the first end of the first L-bracket is removably connected to the clamp, the second end of the first L-bracket having an opening, the opening of the second end of the first L-bracket is configured to receive a first end of a second L-bracket, a second end of the second L-bracket is configured to be received by the at least one opening of the pegboard.

Clause 8. The surgical positioning device of clause 6, wherein the clamp in communication with a ratcheting element with a resilient member.

Clause 9. A method of using a surgical positioning device, comprising: providing a surgical positioning system; providing a table; providing a pegboard, the pegboard overlaying the table; placing a distal end of a user's limb within a boot; and connecting the boot to the pegboard.

### BRIEF DESCRIPTION OF THE DRAWINGS

FIG. 1 depicts a side perspective view of the boot of the novel surgical positioning system, according to at least some embodiments disclosed herein;

FIG. 2 depicts a front perspective view of the boot of the novel surgical positioning system, according to at least some embodiments disclosed herein;

FIG. 3 depicts a rear perspective view of the boot of the novel surgical positioning system, according to at least some embodiments disclosed herein;

FIG. 4 depicts a top perspective view of a distal portion of a user's limb being retained within the boot of the novel surgical positioning system, according to at least some embodiments disclosed herein;

FIG. 5 depicts a top perspective view of a length of material overlaying a distal portion of a user's limb being retained within the boot of the novel surgical positioning system, according to at least some embodiments disclosed herein;

FIG. 6 depicts a side perspective view of the housing hingedly connected to a portion of a t-track of the novel surgical positioning system, according to at least some embodiments disclosed herein;

FIG. 7 depicts a front perspective view of the guide having a t-bolt located opposite a peg, according to at least some embodiments disclosed herein;

FIG. 8 depicts a side view of the guide having a t-bolt located opposite a peg, according to at least some embodiments disclosed herein;

FIG. 9 depicts a perspective view of the t-track receiving the t-bolt of the guide to form an adapter, according to at least some embodiments disclosed herein;

FIG. 10 depicts a top perspective view of the adapter having a side connected to a pegboard, according to at least some embodiments disclosed herein;

FIG. 11 depicts a perspective view of the pin of the boot configured to be received by an opening of the housing of the adapter, the adapter having a side connected to a pegboard, according to at least some embodiments disclosed herein;

FIG. 12 depicts a perspective view of the boot connected to the adapter, the adapter having a side connected to a pegboard, wherein the boot is positioned in an upright orientation, according to at least some embodiments disclosed herein;

FIG. 13 depicts a perspective view of the boot connected to the adapter, the adapter having a side connected to a pegboard, wherein the boot having a rotated orientation, according to at least some embodiments disclosed herein;

FIG. 14 depicts a perspective view of a clamp configured to secure a pegboard to a table with a ratcheting element, according to at least some embodiments disclosed herein;

FIG. 15 depicts a perspective view of the ratcheting element of FIG. 14, according to at least some embodiments disclosed herein;

FIG. 16 depicts a side perspective view of an alternate embodiment of a ratcheting element, according to at least some embodiments disclosed herein;

FIG. 17 depicts a top perspective view of the boot and adapter configured for operative leg placement in adduction, external rotation, with the knee extended, with or without extension at the hip joint, according to at least some embodiments disclosed herein;

FIG. 18 depicts a top perspective view of the boot and adapter configured for operative leg placed into adduction, external rotation, with the knee flexed up to 90 degrees, according to at least some embodiments disclosed herein;

FIG. 19 depicts a front view of a non-operative leg holder, according to at least some embodiments disclosed herein; and

FIG. 20 depicts a perspective view of a non-operative leg holder, according to at least some embodiments disclosed herein.

### DESCRIPTION OF THE PREFERRED EMBODIMENTS

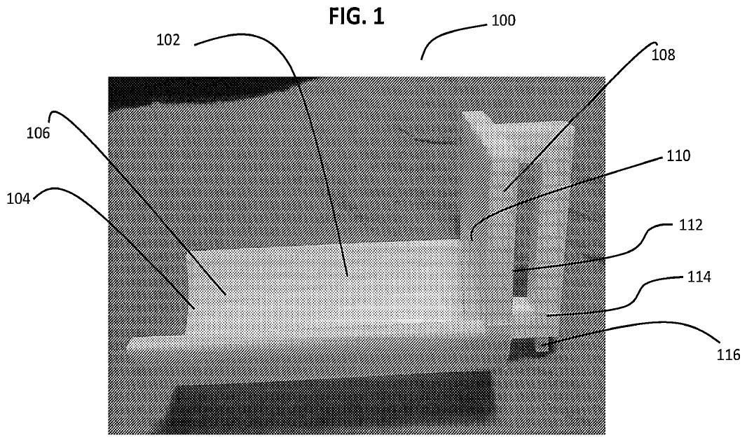

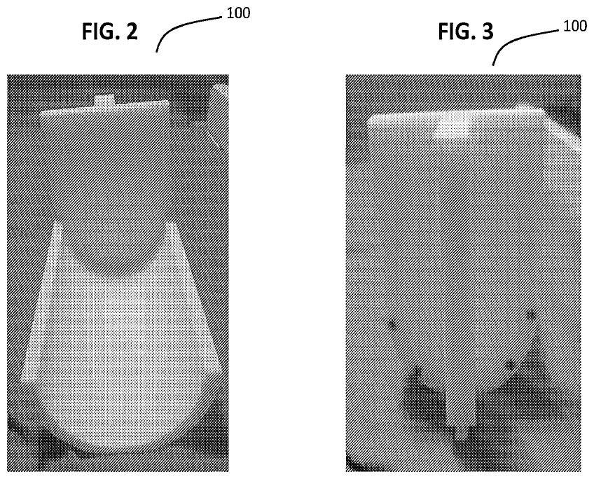

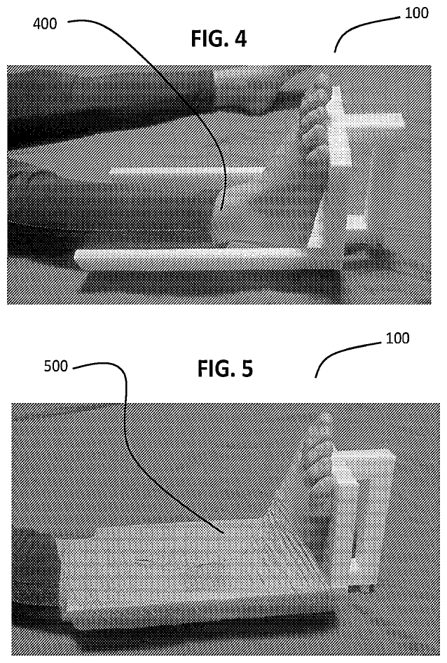

FIGS. 1-5 and 11-13 illustrate boot 100 of the novel surgical positioning system. FIG. 1 shows boot 100 having receiving structure 102 having channel 104. Channel 104 has radiused surface 106 configured to receive a distal portion of limb 400 (FIGS. 4-5) of a user. Boot 100 has plate 108 connected to an end of receiving structure 102. Plate 108 has first side 110 located opposite second side 112. Plate 108 is oriented perpendicular to channel 104 of receiving structure 102. Protrusion structure 114 is connected to second side 112 of plate 108. Protrusion structure 114 has pin 116 extending from a surface therefrom.

FIG. 4 depicts distal portion of user's limb 400 being retained within boot 100 of the novel surgical positioning system.

FIG. 5 depicts length of material 500 overlaying distal portion of a user's limb 400 being retained within boot 100 of the novel surgical positioning system. It is within the scope of this invention for length of material 500 to include, but not be limited to, a bandage configured to be wrapped around user's limb 400 and receiving structure 102 (FIG. 1) of boot 100.

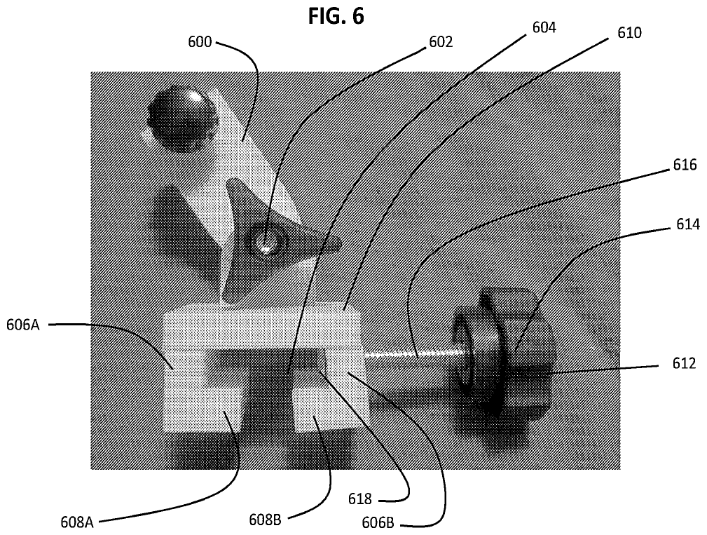

FIG. 6 illustrates housing 600 hingedly pivotal at fulcrum 602. T-track 604 is formed from first protrusion 606A having first flange 608A located opposite second protrusion 606B having second flange 608B. Housing 600 is in communication with outer wall surface 610 of t-track 604. T-track 604 has locking mechanism 612. In an example, locking mechanism 612 is knob 614 connected to threaded rod 616. An end portion of threaded rod 616 has plate 618 connected thereto.

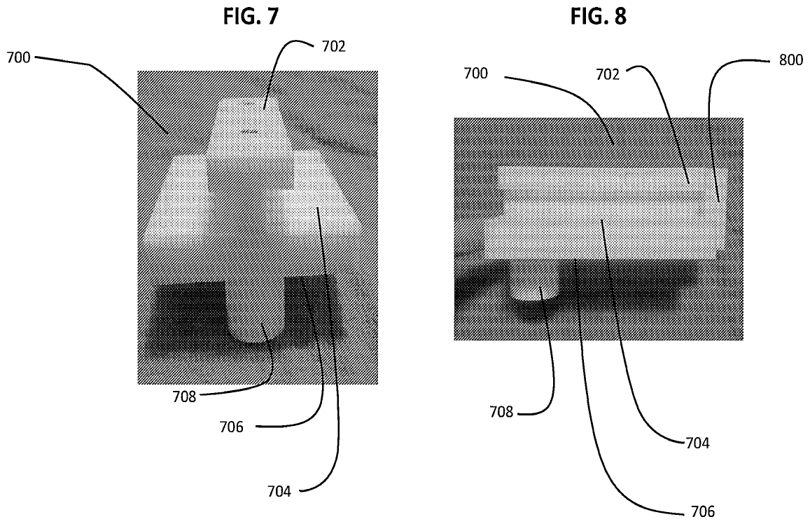

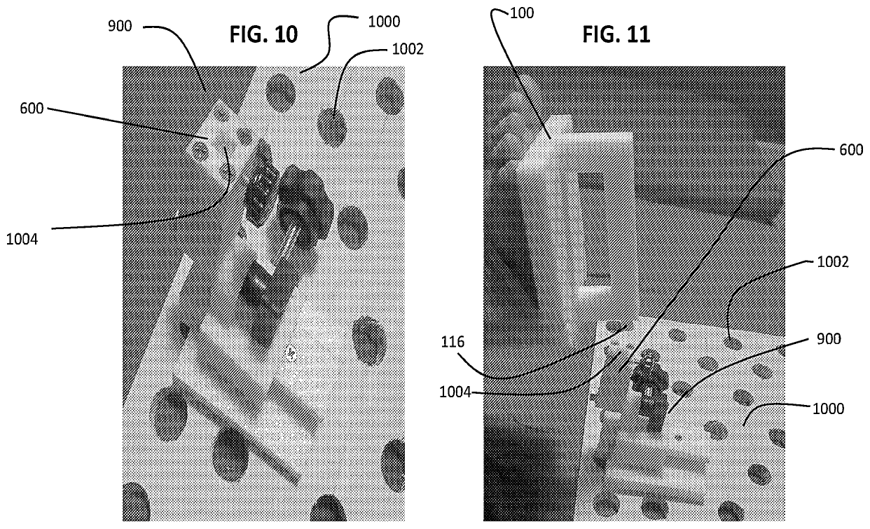

FIGS. 7-8 illustrate guide 700 having first side 704 located opposite second side 706. First side 704 of guide 700 having t-bolt 702. T-bolt 702 is located opposite peg 708. Peg 708 is configured to be received by at least one opening 1002 (FIGS. 10-11) of pegboard 1000 (FIGS. 10-11).

FIG. 8 shows guide 700 having limit structure 800 configured to prevent the t-bolt of the guide from inadvertently exiting the t-track.

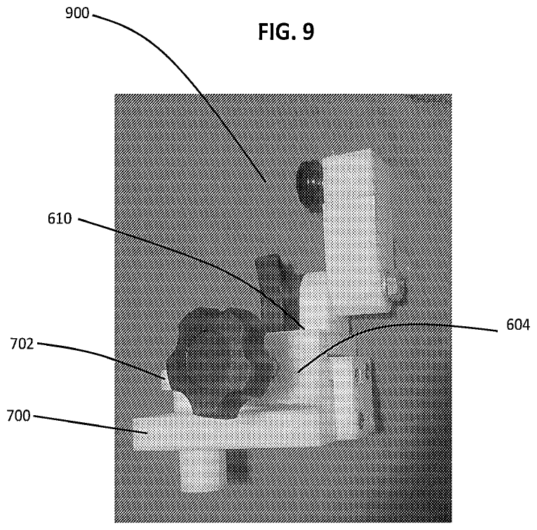

FIG. 9 illustrates adapter 900. T-track 604 receives t-bolt 702 of guide 700 to form adapter 900. T-bolt 702 is configured to slidably traverse a chamber of t-track 604.

FIGS. 10 and 11 illustrate adapter 900 having a side connected to pegboard 1000. Pegboard 1000 has at least one opening 1002. Housing 600 has opening 1004.

FIG. 11 illustrates pin 116 of boot 100 configured to be received by chamber and/or opening 1004 housing 600 of adapter 900.

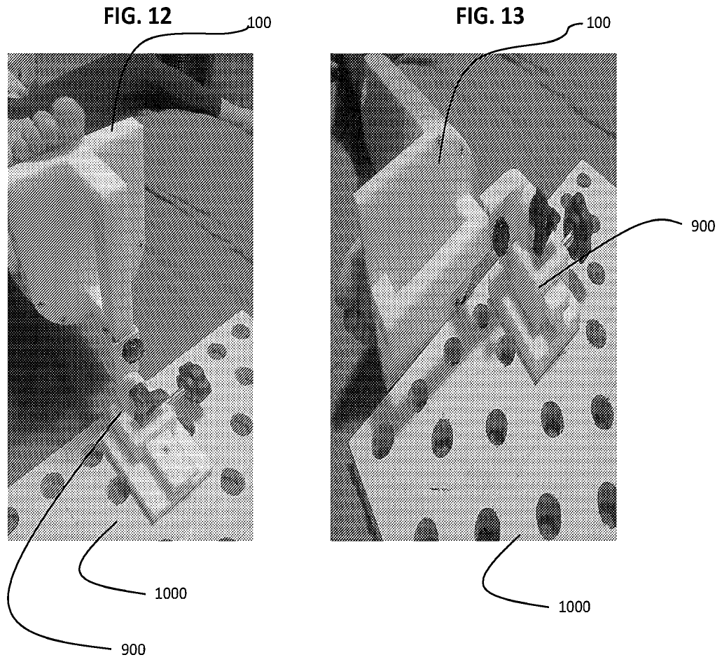

FIGS. 12-13 illustrate boot 100 connected to adapter 900. Adapter 900 having a side connected to pegboard 1000. FIG. 12 shows boot 100 being positioned in a first position being an upright and/or a substantially vertical orientation. FIG. 13 shows boot 100 being positioned in a second position being rotated in a tilted and/or a substantially horizontal orientation.

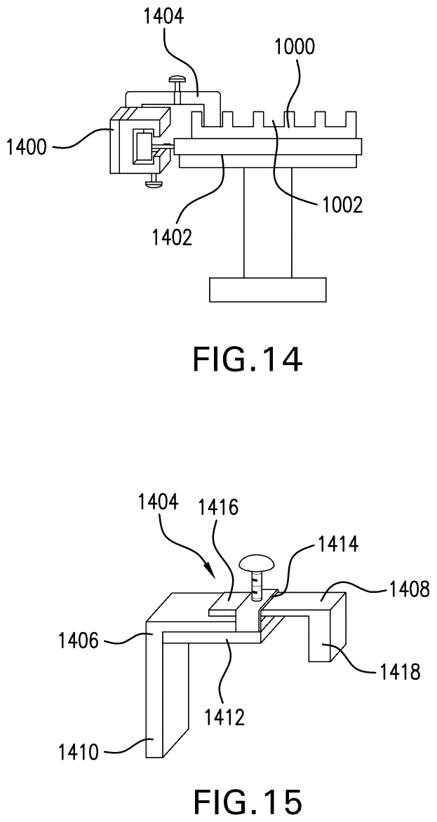

FIG. 14 depicts a perspective view of clamp 1400 configured to secure pegboard 1000 to table 1402 with ratcheting element 1404. Ratcheting element 1404 has an end configured to be received by an opening of clamp 1400. Ratcheting element 1404 has another end configured to be received by at least one opening 1002 of pegboard 1000.

FIG. 15 illustrates ratcheting element 1404 having first L-bracket 1406 having first end 1410 and second end 1412. First end 1410 of first L-bracket 1406 is removably connected to clamp 1400 (FIG. 14). Second end 1412 of first L-bracket 1406 has opening 1414 configured to receive first end 1416 of second L-bracket 1408. Second end 1418 of second L-bracket 1408 is configured to be received by the at least one opening 1002 (FIG. 14) of pegboard 1000 (FIG. 14). It is within the scope of this invention for ratcheting element 1404 to be adjustable in size using a locking mechanism being a fastener. It is within the scope of this invention for adjustable to include, but not be limited to, slidably traversable, telescoping, and/or hingedly pivotable. A fastener may include, but not be limited to, a threaded rod with a knob and/or a gripping member.

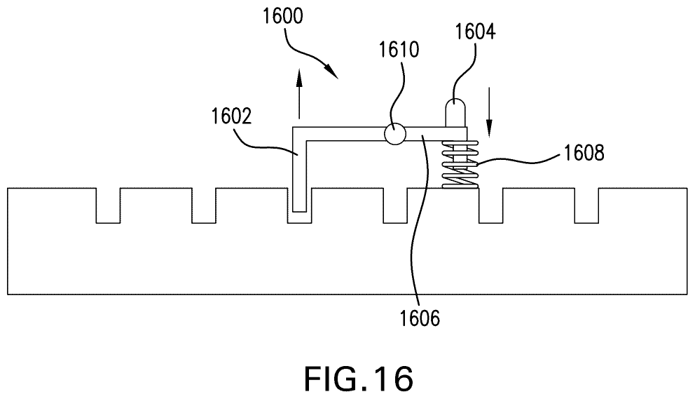

FIG. 16 illustrates an alternate embodiment of ratcheting element 1600. First L-bracket 1602 is oriented to be positioned up and/or down the y-axis when button 1604 of second L-bracket 1606 is depressed. When first L-bracket 1602 is oriented in a down position, second L-bracket 1606 having button 1604 does not depress resilient member 1608 and at least a portion of first L-bracket 1602 is retained within at least one opening 1002 of pegboard 1000. Resilient member 1608, such as a spring, is connected to the second L-bracket. When first L-bracket 1602 is oriented in an up position (not shown), second L-bracket 1606 having button 1604 depresses resilient member 1608 and at least a portion of first L-bracket 1602 is lifted and removed from at least one opening 1002 of pegboard 1000. First L-bracket 1602 is hingedly connected to second L-bracket 1606 at fulcrum 1610.

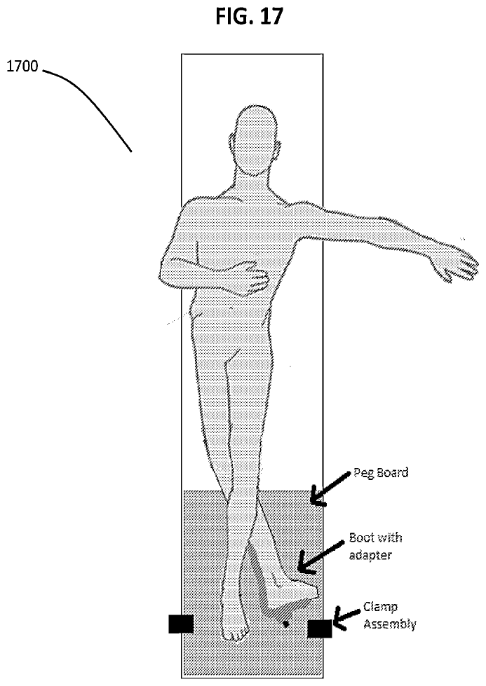

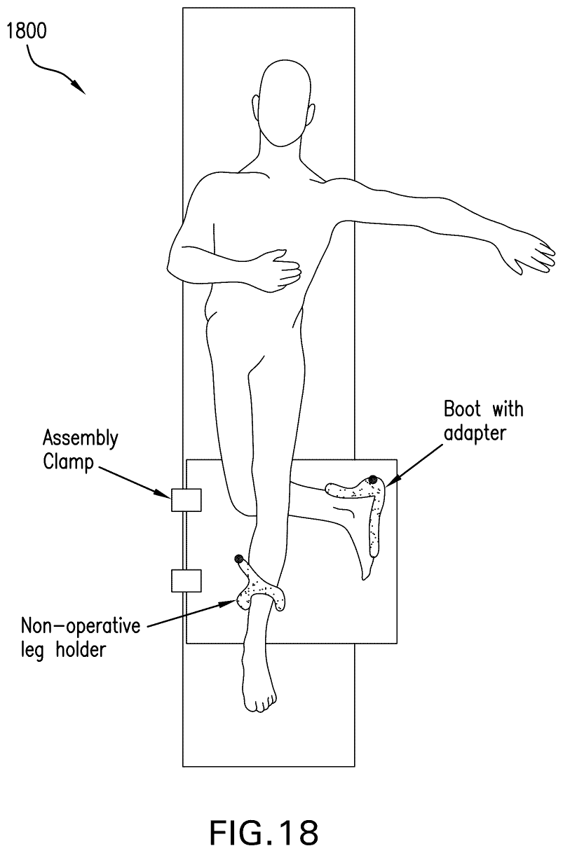

FIG. 17 depicts orientation 1700 of the boot and adapter configured for operative leg placement in adduction, external rotation, with the knee extended, with or without extension at the hip joint. During hip surgical procedures, the extremity may be placed in different positions depending on the exposure needed at the time and surgeon preference. During anterior total hip arthroplasty, the extremity may be placed in several positions for exposure and preparation of the proximal femur. Two commonly used positions include orientation 1700 illustrated in FIG. 17 and orientation 1800 illustrated in FIG. 18. This device would help accommodate these two commonly used positions. The pegboard may be rectangular in nature, so it can be placed with the longest axis parallel with the longitudinal axis of the bed for the orientation 1700, or the longest axis perpendicular to the longitudinal axis of the for orientation 1800. The clamp assembly can be placed in any peg hole, so they could be placed as shown in FIG. 17 or FIG. 18 or any other peg holes depending on surgeon preference and design of the surgical bed.

FIG. 18 depicts orientation 1800 of the boot and adapter configured for operative leg placed into adduction, external rotation, with the knee flexed up to 90 degrees. This is often referred to as the FIG. 4 position, or lazy FIG. 4 if less knee flexion is desired.

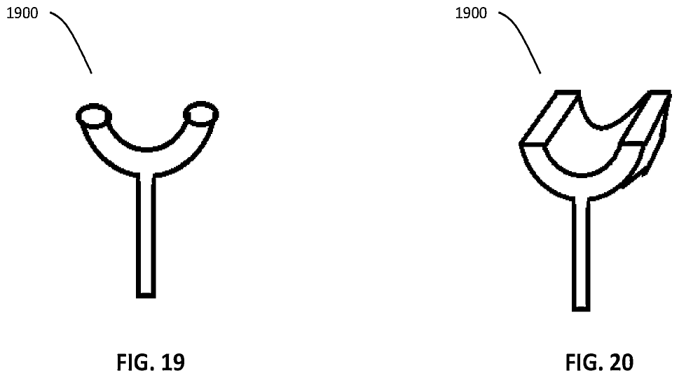

FIGS. 19 and 20 depicts non-operative leg holder 1900. An issue that arises when positioning the surgical leg for exposure of the femur is what to do with the nonsurgical leg. Many times the nonsurgical leg is supported on a padded Mayo stand. A Mayo stand, however, is not sterile. A sterile Mayo cover can be placed, however the base of the Mayo stand and vertical support bar are not sterile and can get uncomfortably close to the surgical field. Therefore, it is within the scope of this invention for a sterile non-operative leg holder as illustrated in FIGS. 18-20 to be used as part of the system to support the nonoperative leg without the added clutter or risk of contamination introduced and using a Mayo stand. Sterile non-operative leg holder 1900 is folding the non-operative left leg elevated so the operative right leg can be placed in FIG. 4 position. Sterile non-operative leg holder 190 would have a base that articulates into any peg hole. The portion of sterile non-operative leg holder 1900 can include a tubular curved or semicircular structure on which the calf of the non-operative leg and rest. Alternatively, other methods of supporting the non-operative leg can be considered including a long semicircular or curved platform, a boot similar to the operative leg boot, a platform which supports the foot only, and/or any combination of these. Additional articulations including but not limited to ball and socket joints can be built where the base of the non-operative leg holder meets the support piece.

### ADDITIONAL DESCRIPTION

A surgical positioning device attaches to an operative table and positions and/or holds the leg to help in the execution surgical procedures such as anterior and anterolateral total hip arthroplasty. The apparatus consists of a boot which is secured to the foot, ankle, lower leg using elastic bandage, nonelastic bandage, straps, or a clamshell design. The boot articulates with a flat board which contains multiple holes for multiple positioning options through an articulating adapter which allows for internal and external rotation of the boot as well as the application and maintenance of traction. The board attaches to the rails of a surgical table with the use of adjustable bed clamps which allow the board to be secured to different surgical tables which may have different widths as well as different pad thicknesses. The adjustable bed clamps also allow the board to be placed more proximal or distal on the surgical table rails to accommodate patient height and positioning.

The boot contains a cut out on the medial and lateral sides to allow for palpation of the medial malleolus, regardless of whether the boot is applied to the left or the right lower extremity. The boot attaches to the articulating adapter through an extension attached on the bottom surface of the boot.

The articulating adapter consists of two main pieces. The first piece, articulating adapter rail, consists of a rail with a cylindrical peg on the bottom. The cylindrical peg on the bottom can be inserted into any of the plurality of openings on the pegboard. Onto the articulating adapter rail attaches the second piece, articulating adapter clamp. This piece connects the boot to the rail piece. The articulation between the boot and the articulating adapter clamp allows for the boot to be internally and/or externally rotated relative to the articulating adapter clamp. The articulating adapter clamp has cut outs to allow internal and external rotation past 90 degrees. The articulating adapter clamp is able to slide proximally and distally on the articulating adapter rail to control the amount of traction on the extremity. The articulating adapter clamp position on the articulating adapter rail is maintained by a mechanism consisting of either: (1) a bolt with a knob that is screwed through the articulating adapter clamp and tightened against the articulating adapter rail, (2) a spring-loaded smooth bolt on the articulating adapter clamp which inserts into indentations at various positions along the rail, or (3) a ratchet mechanism with a spring mechanism which automatically clicks/locks as the articulating adapter clamp is slid along the articulating adapter rail, and releases with a lever which disengages the ratchet mechanism.

The board may include any size and/or shape such as a square or a rectangular board with multiple rows of pegholes to allow various possible positions for insertion of the articulating adapter rail peg. This allows adjustment of lower extremity adduction. The board attaches the rails of the operative table with clamps that have a slot onto which adjustable arms insert. The adjustable arms may be raised and/or lowered along the slot on the bed rail clamps to accommodate varying operative table pad thicknesses. The adjustable arms can also telescope in and out to accommodate varying operative table widths. The end of the adjustable arm has a peg which inserts into an opening of the pegboard in order to secure it to the table.

A surgical positioning device is configured to connect to an operative table and positions/holds the leg to help in the execution surgical procedures such as anterior and anterolateral total hip arthroplasty. The device is meant to be sterile, and applied to the surgical bed after the sterile drapes are placed. The apparatus consists of a boot which is secured to the foot, ankle, lower leg using readily available cohesive elastic bandage. Alternatively, other methods of securing the lower extremity to the boot can be considered such as ratcheting straps or a clamshell design. The boot can be cushioned with a single use sterile foam cushion which are readily available from multiple manufacturers.

The boot allows for palpation of the medial malleolus, regardless of whether the boot is applied to the left or the right lower extremity. The boot attaches to the articulating adapter through an extension attached on the bottom surface of the boot or boot handle. The boot articulates with a flat board which contains multiple holes for multiple positioning options through the articulating adapter complex which allows for internal and external rotation of the boot as well as the application and maintenance of traction. The board attaches to the rails of a surgical table with the use of adjustable positioner stabilizer which allows the board to be secured to different surgical tables which may have different widths as well as different pad thicknesses. The adjustable bed clamps also allow the board to be placed more proximal or distal on the surgical table rails to accommodate patient height and positioning.

The articulating adapter complex consists of two main pieces. The first piece, articulating adapter rail, consists of a rail with a cylindrical peg on the bottom. The cylindrical peg on the bottom can be inserted into any of the pegholes on the board. Onto the articulating adapter rail attaches the second piece, articulating adapter clamp. The articulating adapter clamp slides onto the articulating adapter rail, and the position of the articulating adapter clamp slides on the articulating adapter rail can be set by tightening the knob on the side of the articulating adapter clamp. Alternatively, different mechanisms of setting the position of the articulating adapter clamp slides on the articulating adapter rail can be devised, such as a spring-tensioned ratcheting mechanism built into the area of the side know which automatically locks the position on indentations on the articulating adapter rail and requires pressing of a button to disengage the position.

The articulating adaptor clamp has an attachment site onto which the boot attaches. In this scenario, the square peg on the handle of the boot inserts onto the square opening on the articulating adapter clamp, and is secured by tightening the small knob. Alternatively, other mechanisms of securing the junction such as a spring-loaded push release mechanism can be considered. The articulation adapter clamp has a hinge which can be adjusted by loosening the triangular knob, using the handle on the boot to set rotation, and tightening the triangular knob to maintain rotation. This allows internal and external rotation of the extremity relative to the surface of the bed by over one hundred degrees. Indentations or cutouts may be designed into the articulating adapter clamp in areas where it impinges with the articulating adapter clamp rail in order to further allow for additional internal and external rotation. Traction can be placed by moving the articulating adaptor complex to a new peg hole on the board, and then reattaching the boot to the articulating adaptor complex. Alternatively, if only a few centimeters of traction are needed, the articulating adaptor clamp position of the articulating adapter rail can be changed. The side knob on the articulating adapter clamp is loosened, traction is applied by pulling on the boot handle, sliding the articulating adapter clamp on the articulating adapter rail, and then tightening the knob to maintain this position. A square or rectangular board has multiple rows of pegholes to allow various possible positions for insertion of the articulating adapter rail peg. This allows adjustment of lower extremity adduction/abduction as well as traction.

The preferred embodiments of the present invention will now be described with reference to the drawings. Identical elements in the various figures may be identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an.” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the description is intended to cover all of the generic and specific features of the invention herein described, and all statements of the scope of the invention that, as a matter of language, might be said to fall therebetween.

It will be further understood that the terms “comprises,” “comprising.” “includes,” and/or “including,” when used herein, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

Now that the invention has been described,

What is claimed is:

1. A surgical positioning device, comprising:
a boot, the boot comprising:
a receiving structure, the receiving structure having a channel, the channel having a radiused surface configured to receive a distal portion of a limb of a user;
a plate, the plate is connected to an end of the receiving structure, the plate having a first side located opposite a second side, the plate is oriented perpendicular to the channel of the receiving structure; and
a protrusion structure, the protrusion structure connected to the second side of the plate, the protrusion structure having a pin;
an adapter, the adapter comprising:
a t-track, the t-track is formed from a first protrusion having a first flange located opposite a second protrusion having second flange;
a housing, the housing is hingedly pivotable at a fulcrum, the housing having a chamber, the chamber of the housing is configured to receive the pin of the protrusion structure of the boot;
wherein the housing is in communication with an outer wall surface of the t-track; and
a guide, the guide having a first side located opposite a second side, the first side of the guide having a t-bolt, the t-bolt is configured to slidably traverse the t-track, the second side of the guide having a peg, the peg is configured to be received by an opening of a peg board.

2. The surgical positioning device of claim 1, wherein the t-track having a locking mechanism.

3. The surgical positioning device of claim 2, wherein the locking mechanism is a knob connected to a threaded rod.

4. The surgical positioning device of claim 3, wherein the threaded rod having a plate connected to an end portion.

5. The surgical positioning device of claim 1, further comprising a pegboard, the pegboard having at least one opening configured to receive the peg of the guide of the adapter.

6. The surgical positioning device of claim 5, further comprising a clamp, the clamp is configured to connect the pegboard to at least a portion of a table.

7. The surgical positioning device of claim 6, further comprising a first L-bracket having a first end and a second end, the first end of the first L-bracket is removably connected to the clamp, the second end of the first L-bracket having an opening, the opening of the second end of the first L-bracket is configured to receive a first end of a second L-bracket, a second end of the second L-bracket is configured to be received by the at least one opening of the pegboard.

8. The surgical positioning device of claim 6, wherein the clamp in communication with a ratcheting element with a resilient member.

9. A method of using a surgical positioning device, comprising:
providing a surgical positioning system;
providing a table;
providing a pegboard, the pegboard overlaying the table;
placing a distal end of a user's limb within a boot; and
connecting the boot to the pegboard.
